(19) United States
(12) Patent Application Publication
(10) Pub. No.: US 2001/0043932 A1
(43) Pub. Date: Nov. 22, 2001

(54) Method of inducing cell-mediated protective immunity against HIV using low doses of immunogens

(75) Inventors: Raoul E. Benveniste, Bethesda, MD (US); Gene M. Shearer, Bethesda, MD (US); Mario S. Clerici, Rockville, MD (US)

Correspondence Address: TOWNSEND AND TOWNSEND AND CREW, TWO EMBARCADERO CENTER, EIGHTH FLOOR, SAN FRANCISCO, CA, 94111-3834, US

(73) Assignee: Secretary of the Department of Health and Human Services

(21) Appl. No.: 09/769,223

(22) Filed: Jan. 24, 2001

Related U.S. Application Data
(63) Continuation of application No. 09/321,498, filed on May 27, 1999, now abandoned.
Application No. 09/321,498 is a continuation of application No. 08/899,081, filed on Jul. 23, 1997, now abandoned.
Application No. 08/899,081 is a continuation of application No. 08/250,417, filed on May 27, 1994, now abandoned.

(51) Int. Cl. A61K 39/21

(52) U.S. Cl. 424/208.1

(57) ABSTRACT

A method and composition is disclosed for inducing a protective immunity against HIV by inoculation with immunogens in low doses that are sufficient to induce a sustained cell-mediated response to HIV but below the amount necessary to induce an offsetting humoral response. The immunogens available for use in this method include attenuated forms of the HIV virus, subunits of the HIV virus, inactivated HIV virus and subinfectious doses of live HIV virus, all delivered at low doses. These immunogens can be prepared with-suitable carriers, adjuvants or diluents and administered either intramuscularly, mucosally (e.g., orally), intravenously or subcutaneously. The effectiveness of the initial dose of immunogen can be monitored for the presence of a sufficient cell-mediated response using a T-cell proliferation assay or an interleukin-2 assay and monitored for the lack of offsetting humoral response using commercially available ELISA assays for anti-HIV antibodies. Depending upon the results of the cell-mediated response assays, supplementary or “booster” inoculations may be appropriate.

### BACKGROUND OF THE INVENTION

Acquired Immune Deficiency Syndrome (“AIDS”) is the clinical manifestation of the infection of CD4⁺ helper T-cells and other targets by human immunodeficiency virus (“HIV”), also previously referred to as human T-lymphotropic virus type III (HTLV-III), Lymphadenopathy-associated virus (“LAV”), or AIDS-related virus (ARV) (hereafter collectively “HIV”). AIDS is a transmissible disease characterized by opportunistic infections and certain malignancies.

One of the major goals of AIDS research is the development of an efficacious vaccine providing broad, long-lasting protection against HIV. Such a vaccine would activate specific immune defenses in the body to protect against a subsequent exposure to an otherwise infectious dose of HIV.

The are two basic modes of specific immunity that can be generated by the immune system in response to an antigen: (1) humoral immunity and (2) cell-mediated immunity (“CMI”). The humoral immune response is based upon the secretion by plasma cells of antibodies which travel to the bloodstream and circulate throughout the body. These antibodies bind to free-floating antigens in order to disarm them and tag them for disposal by macrophages. Some of these antibody-antigen complexes also activate complement proteins circulating in the blood. The complement proteins then trigger other aspects of the immune response, including the release of histamine which causes inflammation.

The components of a cell-mediated immune response include killer T-cells, performs, macrophages, CD4⁺ T-helper cells that mediate delayed-type hypersensitivity (“DTH”) and CD8⁺ cytotoxic T lymphocytes (“CTL”). In a cell-mediated response to a viral infection, killer T-cells will bind to the MHC-1 antigen complex on infected cells that are producing copies of the virus. There, the killer T-cells release proteins called performs, which cause pores to form in the infected cell's outer membrane admitting toxic substances which eventually kill the cell. The killer T-cells are unharmed by the perforins so they can subsequently detach and move on to target other infected cells.

The mechanisms which determine whether a humoral or cell-mediated response will be invoked by various antigens have become clearer with the identification of distinct CD4⁺ T-helper cell subsets. These CD4⁺ T-helper cell subsets are categorized by-their different functions and by the constellation of cytokines that they produce. T-helper cells of type 1 (“T_{H}1”) secrete interferon γ (“IFN-γ”) and interleukin-2 (“IL-2”). They also contribute to cell-mediated responses such as DTH and macrophage activation. T-helper cells of type 2 (“T_{H}2”) produce IL-4, IL-5 and IL-10 and thereby help B cells generate antibody responses.

There is a tendency for either the cell-mediated or the antibody response to predominate in any particular immune response [J. Salk, P. Bretscher, P. Salk, M. Clerici, G. Shearer, Science 260, 1270 (1993); T. R. Mosmann and R. L. Coffman, Adv. Immunol. 46, 111 (1989); P. A. Bretscher, Cell Immunol. 13, 171 (1974); I. A. Ramshaw, P. A. Bretscher, C. R. Parish, Eur. J. Immunol. 6, 674 (1976); P. Salgame et al., Science 254,-279 (1991)]. This tendency is thought to result from cross-regulation by T-helper cells. For example, T_{H}1 cells (or other coordinately induced cells) are thought to inhibit the induction of T_{H}2 humoral responses through production of IFN-γ cytokines. By contrast, T_{H}2 cells (or other coordinately induced cells) are thought to inhibit the generation of T_{H}1 cell mediated responses through the production of such cytokines as IL-4 and IL-10.

This tendency for either the humoral or cell-mediated response to predominate is believed to apply to HIV infection and AIDS. Peripheral blood mononuclear cells (“PBMC”) from antibody negative, but HIV exposed, individuals respond to HIV envelope antigens with a T_{H}1 response, that is, they produce IL-2 [M. Clerici, J. A. Berzofsky, G. M. Shearer, C. O. Tacket, J. Infect. Dis. 164, 178 (1991); G. M. Shearer et al., J. Cell Biochem. Suppl. 16E, 112 (1992)]. Moreover, as asymptomatic HIV antibody positive individuals progress towards AIDS, their peripheral blood lymphocytes shift from a T_{H}1-predominant to a T_{H}2-predominant pattern of cytokine production [M. Clerici et al. J Clin. Invest. 91, 759 (1993); G. M. Shearer and M. Clerici, Chem. Immunol. 54, 21 (1992); M. Clerici and G. M. Shearer, Immunol. Today 14, 167 (1993)].

Attempts to develop a vaccine to prevent infection with HIV generally have concentrated on the elicitation of specific virus-neutralizing antibodies. A region-of the HIV surface coat protein (gp120) which is involved in the generation of such antibodies has been defined [Goudsmit et al., Proc. Natl. Acad. Sci. U.S.A. 85, 4478 (1988); Ho et al., J. Virol. 61, 2024 (1987); Matsushita et al., J. Virol. 62, 2107 (1988); Palker et al., Proc. Natl. Acad. Sci. U.S.A. 85, 1932 (1988) Rusche et al., Proc. Natl. Acad. Sci. U.S.A. 85, 3198 (1988); Skinner et al., J. Virol. 62, 4195 (1988)]. However, attempts to use the intact viral coat protein or portions thereof to readily elicit sufficient levels of neutralizing antibodies to protect against infection have proven unsuccessful [Berman et al., Proc. Natl. Acad. Sci. U.S.A. 85, 5200 (1988); Hu et al., Nature 328, 721 (1987); Lasky et al., Science 233, 209 (1986); Putney et al., Science 234, 1392 (1986); Robey et al., Proc. Natl. Acad. Sci. U.S.A. 83, 7023 (1986); Rusch et al., Proc. Natl. Acad. Sci. U.S.A. 84, 6924 (1987)].

An object of this invention is the development of a vaccination method and composition which activates a protective cell-mediated response to HIV but avoids reducing that response through the activation of an offsetting humoral response. Applicants have found through their work in macaques with a virus related to HIV, the simian immunodeficiency virus (“SIV”), that this object can be accomplished through a method of administering low doses of immunogens. In their studies, applicants have found that administration of high doses of SIV mucosally (intrarectally) to macaques results in infection and antibody production with minimal cell-mediated immunity. By contrast, administration of lower doses elicits strong and long-term protective cell-mediated immunity with neither antibody production nor detectable infection [M. Clerici et al., IX International Conference on AIDS (Berlin, 7 to 11 June, 1993), abstract 3279].

### SUMMARY OF THE INVENTION

The present invention provides a method and composition of inducing a protective immunity against HIV by inoculation with immunogens in doses sufficient to induce a cell-mediated response to HIV but below the amount necessary to induce an offsetting humoral response. The immunogens available for use in this method include attenuated forms of the HIV virus, subunits of the HIV virus, inactivated HIV virus and subinfectious doses of live HIV virus, all delivered at low doses. These immunogens can be prepared with suitable carriers, adjuvants or diluents and administered either intramuscularly, mucosally (e.g., orally), intravenously or subcutaneously. The effectiveness of the initial dose of immunogen can be monitored for the presence of a sufficient cell-mediated response using a T-cell proliferation assay or an interleukin-2 assay and monitored for the lack of an offsetting humoral response using commercially available ELISA assays for detecting anti-HIV antibodies. Depending upon the results of the cell-mediated response assays, supplementary or “booster” inoculations may be appropriate.

### BRIEF DESCRIPTION OF THE DRAWINGS

FIG. 1 conceptually illustrates the different effects upon subsequent challenge with HIV of vaccination using high and low doses of immunogens [J. Salk, P. Bretscher, P. Salk, M. Clerici, G. Shearer, Science 260, 1270 (1993)].

ABBREVIATIONS AND DEFINITIONS

| | |
|---|---|
| Ab: | Antibody |
| Adjuvant: | Any compound which enhances the desired |
| | response by the body to a pharmaceutical. |
| AIDS: | Acquired Immune Deficiency Syndrome |
| Antibody: | A large defense protein, synthesized by the B |
| | Lymphocytes, composed of four proteins linked |
| | together in a “Y” shaped bundle. |
| Antigen/ | Any large molecule whose entry into the body |
| Immunogen: | provokes an immune system response |
| Attenuated | A virus which has been modified to no longer |
| Virus: | be pathogenic |
| CMI: | Cell Mediated Response |
| Cytokines: | A class of proteins derived from T-Cells which |
| | help regulate the immune system |
| ELISA: | Enzyme Linked Immunosorbent Assay |
| IL-2: | Interleukin-2, a lymphokine secreted by |
| | stimulated helper-T Cells which promotes the |
| | proliferation/differentiation of more helper T- |
| | Cells to combat an infection. |
| MHC: | Major Histocompatibility Complex |
| Perforin: | A 70 kd protein which lyses infected cells by |
| | polymerizing to form transmembrane pores 100 |
| | A wide. The pores burst the cell by allowing |
| | ions to rush into the cell (by osmotic |
| | pressure) through the pores. |

### DESCRIPTION OF THE SPECIFIC EMBODIMENTS

The present invention provides an effective method of vaccinating against HIV by using low doses of immunogen to invoke a strong cell-mediated response, while avoiding an offsetting humoral response.

### A. Preparation and Selection Of The Immunogen

The present invention can be practiced with a number of immunogenic variants of the HIV virus. These immunogenic variants include attenuated forms of the HIV virus, subunits of the HIV virus, inactivated HIV viruses and subinfectious amounts of live HIV virus. In selecting such an immunogen, the objective is to provoke a cell-mediated immune system response to the immunogen which will later be effective, if necessary, in protecting against challenge by higher (infectious) amounts of live HIV. At the same time, it is important in selecting an immunogen not to infect the patient with HIV.

A first class of immunogens for the present invention are attenuated viruses. Attenuation refers to the production of virus strains which have essentially lost their disease producing ability. Suitable forms of attenuated HIV include, for example, HIV that has been recombinantly modified by DNA insertions, deletions or substitutions to its genome at critical points. Such an attenuated form of HIV would include HIV with all or a portion of the nef open reading frame deleted. In studies with rhesus monkeys using SIV, it was discovered that the presence of nef in the virus was required for maintaining high virus loads during the course of persistent infection in vivo and for full pathologic potential. Nonetheless, deleting nef in the virus did not prevent the virus from replicating [H. W. Kestler, III et al., Cell 65, 651 (1991); Daniel et al., Science 258, 1938 (1992)]. As a result of these studies, it is likely that the immune system would react to HIV having a nef deletion in the same way that it would react to normal HIV, yet without the same risk of infection.

A nef-deleted HIV strain can be constructed using standard recombinant techniques. Suitable techniques are described, for example, in Sambrook et al., Molecular Cloning: A Laboratory Manual, 2nd ed., Cold Spring Harbor Laboratory Press, 1989, Chapter 15, which is incorporated by reference. In one preferred approach, a nef-containing fragment of an HIV cDNA clone is isolated and subcloned into a vector that replicates in a single-stranded form, such as the bacteriophage M13mp18. A suitable HIV proviral clone, BH10, is available from the American Type Culture Collection, ATCC Accession No. CRL 40125. One can identify an appropriate fragment by referring to the HIV nucleotide sequence presented in, for example, Ratner et al., Nature 313: 277-284 (1985) and Wain-Hobson et al., Cell 40: 9 (1985).

An oligonucleotide primer is synthesized which is. complementary to the two regions of the nef gene fragment that flank the desired site of deletion. The oligonucleotide sequence can be deduced by reference to the published HIV nucleotide sequences listed above. This primer is annealed to the isolated, single-stranded subclone that contains the nef gene fragment. The primer is extended using a DNA polymerase and ligase is used to circularize the resulting second strand.

The resulting hybrid plasmid contains one strand with the mutation and one wild-type strand. A plasmid that contains the mutation in double-stranded form is obtained using techniques known to those skilled in the art. For example, the hybrid plasmid can be transformed into E. coli, allowed to replicate, and those colonies that contain only the deletion mutant identified by an appropriate screening technique.

Alternatively, the wild-type strand can be removed from the hybrid plasmid before transformation. Several methods for accomplishing this are known to those skilled in the art, such as the method described by Eckstein's group [Taylor et al., Nucl. Acids Res. 13: 8764-8785 (1985); Nakamaye et al., Nucl. Acids Res. 14: 9679-9697 (1986); Sayers et al., Biotechniques 13: 592-596 (1992)] in which the primer extension is performed in the presence of a thionucleotide. After this primer extension, the hybrid plasmid is digested with a restriction enzyme that does not cleave a DNA strand at a position where a thionucleotide is incorporated (e.g., NciI). The wild-type strand is nicked by the restriction enzyme. Exonuclease III is then utilized to degrade most of the wild-type strand, including the portion complementary to the original oligonucleotide primer. The remaining portion of the wild-type strand serves as a primer for synthesis of a complete second strand using the deleted strand as template. The Amersham SCULPTOR™ Oligonucleotide-Directed in vitro Mutagenesis System, Version 2 kit from Amersham, Inc. (Arlington Heights, Ill.) is especially useful for this method of mutagenesis. One of skill in the art will recognize that other methods are available for constructing deletion or other mutants that do not produce functional nef (e.g., nonsense and frameshift mutations). For example, polymerase chain reaction can be used to construct suitable mutants.

Following the deletion mutagenesis, the nef-containing fragment from which part of the nef coding region has been deleted is excised from the plasmid, isolated, and cloned into the HIV proviral clone at the appropriate location. The modified proviral clones are purified, checked for substitution of the deleted nef fragment using restriction mapping, DNA sequencing, or other techniques and used to infect human T-cell lines.

Attenuation of HIV can, of course, also be to accomplished by other well known techniques such as subjecting the virus to unusual growth conditions (e.g., heat or cold sensitivity) and/or frequent passage in cell culture. Viral mutants are then selected which have lost virulence, yet are capable of eliciting an immune response. Many attenuated viruses make good immunogens since they actually replicate in the host cell and elicit long-lasting immunity. However, care must be taken to make sure the attenuation is complete. Insufficient attenuation and improper administration of the attenuated vaccine can lead to inadvertent infection.

A second class of immunogens useful for the present invention are subunits of HIV. Such subunits are polypeptide components of HIV which are capable of eliciting an immune response. For HIV, the envelope proteins gp120 and gp160, as well as the internal p24 gag protein, are especially useful as subunit immunogens, although other HIV polypeptides or fragments of polypeptides may also be useful. Such subunits can be isolated from whole HIV using conventional techniques, such as lysis, affinity chromatography, high pressure liquid chromatography (“HPLC”), chemical synthesis or enzymatic synthesis. Techniques for solid phase chemical synthesis of polypeptide subunits are described, for example, in Merrifield, J. Amer. Chem. Soc. 85:2149-2156 (1963), which is incorporated by reference. Such chemical synthesis, though, is generally employed only for the production of polypeptides of fewer that 100 amino acids.

A preferred method for producing such polypeptide subunits involves expression in host cells of recombinant DNA molecules encoding the desired polypeptide subunit. Techniques for such recombinant expression are now well known and consist generally of: (a) isolation of a gene, or gene fragment, encoding the desired HIV viral protein, (b) insertion of the gene or gene fragment into an expression vector, (c) identification and growth of the recombinant expression vector in a host system which is capable of replicating and expressing the gene, (d) identification and purification of the gene product and (e) determination of the immunopotency of the product.

A number of host systems are available for the expression of subunit polypeptides. These host systems include yeast, filamentous fungi, insect (especially employing baculoviral vectors) and mammalian cells, as well as bacterial systems. In such host systems, the gene or gene fragment will typically be operably linked to a promoter in an expression vector. Of course, viral proteins expressed in prokaryotic host systems will be in a nonglycosylated form while viral proteins expressed in eukaryotic host systems are often glycosylated. For this reason, mammalian or insect host systems are preferred because the protein folding, transport and processing (including glycosylation) more closely approximate what occurs in an infected human cell.

A third class of immunogen for use in the present invention is inactivated or “killed” HIV. Inactivation of HIV renders it harmless as an infectious biological agent but does not destroy its immunogenicity. HIV can be inactivated in a number of ways, including sufficient exposure to various chemical solutions (e.g., betapropiolactone, formalin, ethylmethanesulfonate, phenol, psoralens, platinum complexes, etc.), heat, ultraviolet (UV) light or ozone.

HIV can also be inactivated by removing key nucleotide sequences from those genes responsible for the replication of the virus. In one example of this type of genetic inactivation, non-infectious HIV is obtained by using in vitro mutagenesis to delete part or all of the portion of the gag nucleocapsid gene that encodes the “zinc finger” region. The gaq protein of HIV and all other known retroviruses contain one or two copies of the following invariant cysteine array which binds zinc ions: -Cys-(X)₂-Cys-(X)₄-His-(X)₄-Cys-. During viral assembly and in the mature infectious virus, the side chains of the invariant residues bind zinc ions to form specific three-dimensional peptide conformations described as retroviral “zinc fingers.” Mutations in either cysteine array of HIV result in virus particles that are completely non-infectious in vitro and package reduced amounts of viral RNA. Similarly constructed mutations of SIV have been shown to be non-infectious in vivo during applicants' work with macaques. By deleting part or all of one or both of these cysteine arrays, one can obtain a non-infectious HIV particle that is useful in the present invention.

One of skill in the art can construct a suitable zinc finger deletion mutants using the mutagenesis techniques previously described for construction of the nef deletion mutants. Here, however, a fragment of the proviral clone that contains the gag gene zinc finger region (rather than the nef gene region) is subcloned and used for the in vitro mutagenesis. Again, an appropriate oligonucleotide primer sequence can be deduced by examination of the published HIV nucleotide sequences [Ratner et al., Nature 313: 277-284 (1985) and Wain-Hobson et al., Cell 40: 9 (1985)]. After the mutated sequence is verified using well-known sequencing techniques, the mutated sequence can be substituted into a proviral clone in place of the full gag gene using appropriate restriction endonucleases. As with the nef-attenuated virus, the modified proviral clones are purified, checked for the correct mutation and then grown in suitable human T-cell lines.

A fourth class of immunogen for the present invention are live viruses administered in subinfectious amounts. Such live viruses can be grown in a T-cell line in the presence of cell culture media. The viruses can then be harvested by pelleting the cells from centrifugation, filtering the supernatant, diluting the virus particles to the desired concentration and then freezing the virus preparation.

### B. Formulation of the Vaccine

Formulation of the vaccine begins with determining an appropriate dose of immunogen. In making this formulation, the most important concern is avoiding inadvertent infection. After addressing this concern, a dosage must be chosen which will invoke a protective cell-mediated response and avoid an offsetting humoral response. To the extent error is made in formulating a dose, such error should be made on side of delivering too little immunogen. In that way, both inadvertent infection and an offsetting humoral response can be avoided. To the extent that a dose is too small to elicit a sufficient cell-mediated response, booster vaccinations can later be given to increase the level of cell-mediated response.

Since use of live HIV as an immunogen is the most critical case for selecting a proper dosage, it is a natural starting point for analysis. Determination of a therapeutically acceptable dose of live HIV immunogen requires an understanding of the total viruses present in such a dose. A preferred manner of measuring the quantity of such viruses is with a “Coulter HIV-1 p24 Antigen Assay” produced by the Coulter Corporation of Hialeah, Fla. This Coulter assay is an enzyme immunoassay which uses microwell strips coated with a murine monoclonal antibody against the HIV p24 core antigen. The Coulter assay detects HIV antigens in plasma, serum or tissue culture media. If present, the antigen binds to the antibody-coated microwells. The bound antigen is recognized by biotinylated antibodies to HIV which react with conjugated streptavidin-horseradish peroxidase (“SA-HRPO”). Color develops from the reaction of the peroxidase with hydrogen peroxidase in the presence of tetramethylbenzidine (“TMB”) substrate. The reaction is terminated by the Coulter Stopping Reagent (“CSR-1”) and the intensity of the color developed is directly proportional to the amount of HIV antigen present in the plasma, serum or tissue culture media.

The instructions for this Coulter assay, which are incorporated by reference, teach how to construct a standard concentration curve which correlates the optical density or color of the microwells with the amount of HIV p24 antigen. From this measurement of p24 antigen, applicants were able to calculate the corresponding concentration of viruses using data generated from sucrose banding and electron microscopy. Applicants first established with sucrose banding that over 90% of the p24 measured by their Coulter assay is actually embodied in viruses, as opposed to being non-virus associated (i.e., nearly all the p24 banded with the viruses at 1.16 grams/cm³). With the knowledge that nearly all of the p24 measured in the Coulter assay is embodied in actual viruses, the concentration of viruses in a measured sample was physically counted with an electron microscope. This physical count of viruses was compared with the Coulter assay concentration for the same sample. This comparison showed that 1 pg (picogram)/ml of p24 measured using the Coulter assay corresponds to approximately 10⁴ viruses/ml. This correlation can also be shown by measuring the number of gag nucleocapsid proteins that bind to a known amount of single stranded RNA. R. L. Karpel et al., J. Biol. Chem. 262, 4961-4967 (1987). Since HIV encodes equimolar amounts of gag nucleocapsid proteins and p24 proteins, the number of p24 proteins produced by HIV can be readily calculated from this measurement. L. E. Henderson et al, J. Virology 66, 1856-1865 (1992).

In their work with SIV in macaques, applicants also found that not all viruses were capable causing infection. In fact, applicants found that, on the average, it required at least 10⁴ viruses in an intravenously administered dose to result in an infection. In other words, applicants have empirically determined that one infectious dose corresponds to 10⁴ viruses.

Applicants' findings and calculations can be presented in tabular form as follows:

| | | |
|---|---|---|
| Coulter Assay p24 gag | total HIV/SIV | infectious |
| antigen concentration | viruses | HIV/SIV doses |

| | | | |
|---|---|---|---|
| 100-500 | ng/ml (nanogram) | 10⁹/ml | 10⁵/ml |
| 10-50 | ng/ml | 10⁸/ml | 10⁴/ml |
| 1-5 | ng/ml | 10⁷/ml | 10³/ml |
| 100-500 | pg/ml (picogram) | 10⁶/ml | 10²/ml |
| 10-50 | pg/ml | 10⁵/ml | 10¹/ml |
| 1-5 | pg/ml | 10⁴/ml | 1/ml |
| 100-500 | fg/ml (femtogram) | 10³/ml | 0.1/ml |
| 10-50 | fg/ml | 10²/ml | 0.01/ml |
| 1-5 | fg/ml | 10/ml | 0.001/ml |
| 100-500 | ag/ml (attogram) | 1/ml | 0.0001/ml |

Based upon this correlation, a 1 ml sample of live HIV immunogen would need to have a p24 gag antigen concentration from the Coulter assay of at least 1 to 5 pg/ml in order to be considered infectious (i.e., one infectious dose). If the 1 ml sample had a lower p24 gag antigen concentration from the Coulter assay, this correlation table, based upon applicants' experiments, indicates that it would not result in infection.

In the case of live HIV immunogens, the vaccine of the present invention should be formulated to be well below the level considered to be infectious. For example, if a 1 ml solution of live HIV immunogen is used in the vaccine, it should have a concentration of p24 gag antigen using the Coulter assay of less than 1 to 5 fg/ml (i.e., 0.001 infectious doses per ml).

Since the risk of infection with attenuated, subunit and inactivated HIV is substantially less than with live HIV, higher doses can be used for those immunogens. For example, higher doses of, for example, 10 to 500 fg measured on the Coulter assay would be appropriate for live, attenuated HIV. Since subunit and inactivated HIV are not live and replicating, appropriate doses could easily range from 10 fg to 20 μg. To the extent that these non-infectious immunogens are used in quantities greater than the infectious dose level (i.e., 1 to 5 pg/ml), though, care must be taken to avoid triggering an offsetting humoral response and thereby undermining the purpose of the present invention.

As with other types of vaccines, the immunogens of the present invention can be combined with carriers or adjuvants, where appropriate, to formulate a therapeutically effective dose. The primary purpose of carriers and adjuvants are to stimulate the immune system. In the case of carriers, this purpose can be accomplished by conjugating multiple copies of the immunogen to a single larger carrier protein. Examples of carrier proteins are Keyhole limpet hemocyanin (KLH), ovalbumin, serum albumin from any mammalian species, globulins such as betalactoglobulin, oxygen-transporting proteins such as hemoglobins, or subunits thereof such as myoglobins, hemocyanins and the like.

Examples of adjuvants are aluminum hydroxide, aluminum phosphates, aluminum potassium sulfate (alum), beryllium sulfate, silica, kaolin, carbon, water-in-oil, emulsions, oil-in-water emulsions, muramyl dipeptide, bacterial endotoxin, lipid X, Corynebacterium parvum (Propionibacterium acnes), Bordetella pertussis, polyribonucleotides, sodium alginate, lanolin, lysolecithin, vitamin A, saponin, liposomes, levamisole, DEAE-dextran, blocked copolymers and other synthetic adjuvants. Adjuvants can be obtained from companies such as Merck and Company, Inc. of Rathway, N.J. (e.g., Merck Adjuvant 65) and Difco Laboratories of Detroit, Mich. (e.g., Freund's Incomplete and Complete Adjuvant).

Since the purpose of carriers and adjuvants in most vaccines is to enhance the humoral response, great care must be taken in using carriers and adjuvants with the present invention because a humoral response is not desired. For this reason, in many applications of the present invention, carriers and adjuvants are to be avoided.

With such small quantities of immunogens used in the present invention, the vaccine should be formulated with one or more appropriate diluents. Such appropriate diluents include water, buffered water, 0.4% saline or a 0.3% glycine solution. These compositions can be sterilized using conventional sterilization techniques and may contain pharmaceutically acceptable auxiliary substances to adjust pH, buffer or adjust toxicity. Examples of such auxiliary substances include sodium acetate, sodium chloride, potassium chloride, calcium chloride and sodium lactate.

### C. Administration of the Vaccine

The vaccine of the present invention is preferably administered in a therapeutically effective amount either intramuscularly, mucosally (e.g., orally), intravenously or subcutaneously. Such a vaccine is most useful for persons at risk for AIDS, but who have not yet been exposed to HIV. Nonetheless, the present vaccine may also be useful for persons exposed to HIV but who are not yet seropositive for HIV antibodies. In the case of such persons, the present vaccine might be useful in boosting their cell-mediated immune response to either prevent infection or delay the onset of AIDS symptoms.

A typical pharmaceutical composition of the present invention for initial inoculation might be made up 1 ml of physiologic saline and 10 to 500 femtograms of nef deleted HIV immunogen as measured by a Coulter p24 gag antigen assay. Such inoculation might be delivered between 1 and 3 times over the course of several months. Actual methods for preparing parenterally administrable compositions will be known or apparent to those skilled in the art and are described in more detail in, for example, Remington's Pharmaceutical Science, 15th ed., Mack Publishing Company, Easton, Pa. (1980), which is incorporated by reference.

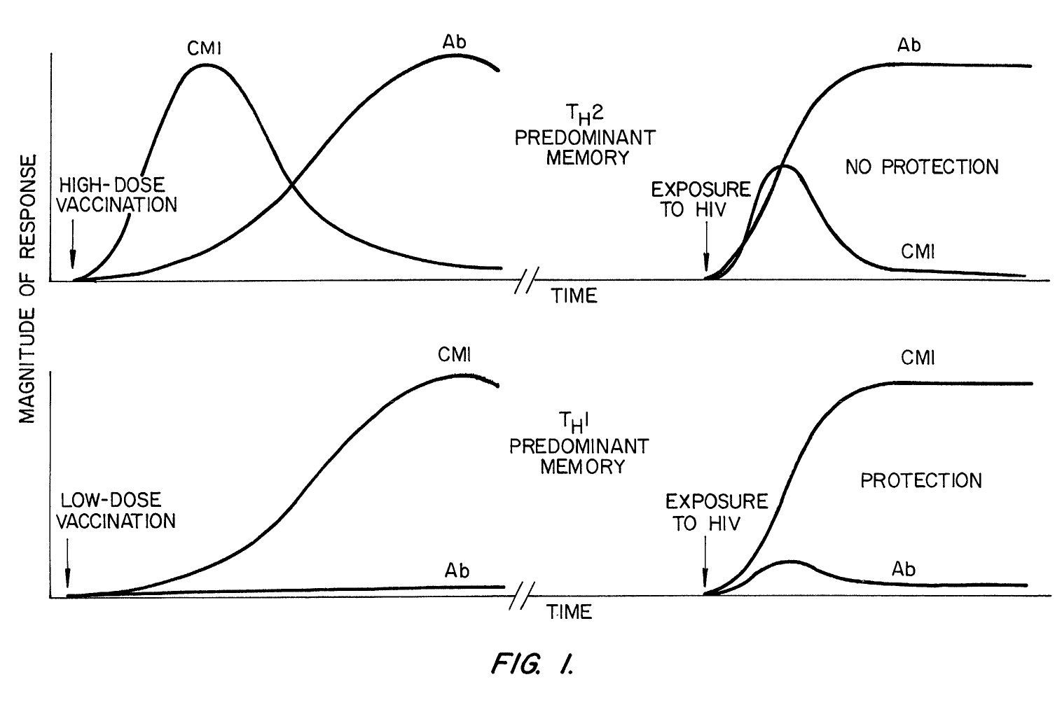

FIG. 1 conceptually illustrates the operation of the present invention as compared with conventional vaccination techniques. For conventional high dose vaccinations as shown in the upper portion of FIG. 1, a transient cell-mediated immune response (CMI) is initially triggered which then gives way to a humoral response. Since the humoral response predominates for such conventional vaccination techniques, there is little CMI protection present to protect against subsequent challenge. By contrast, in the present invention as shown in the lower portion of FIG. 1, a low dose vaccination is given which is sufficient to trigger a CMI response but insufficient to trigger an offsetting humoral response. Upon subsequent challenge, the CMI response will still be present to provide protection.

As previously noted, it is best to err on the side of including too little immunogen in the vaccine formulations rather than too much. This is particularly true when using live HIV as an immunogen. After each inoculation, the effectiveness of the vaccine to invoke a cell-mediated response and avoid an offsetting humoral response can be tested. To the extent that the cell-mediated response is not sufficiently strong from previous inoculations, a supplemental or “booster” vaccine can be formulated using the assay results.

### E. Testing the Effectiveness of the Vaccine

The vaccine of the present invention can be assayed both for the presence of a cell-mediated response and for the lack of a humoral response.

The presence and strength of a cell-mediated response may preferably be tested using either an HIV antigen-stimulated T-cell proliferation assay or an interleuken-2 (IL-2) production assay. In both cases, peripheral blood mononuclear cells (“PBMC”) need to be isolated from vaccinees both before and after inoculation. For the T-cell proliferation assay, PBMC from the vaccinees are cultured for approximately six days in at least five different samples with media containing human plasma and separate HIV antigens. Suitable antigens for such a T-cell proliferation assays are HIV envelope proteins (e.g., gp120, gp160) as well as epitopes of such envelope proteins [e.g., T1, T2, Th4, P18-IIIB and P18-MN; see, Clerici et al., J. Immunol. 146, 2214 (1991); Clerici et al., J. Infect. Dis. 164, 178 (1991); Berzofsky et al., Nature 334, 706 (1988)]. Nonetheless, to the extent that epitopes are used to test the cell-mediated effectiveness of a subunit vaccine, it is important to select epitopes that are encompassed within the subunit (i.e., using epitopes of gp120 to test the effectiveness of a gp120 subunit vaccine). After the six day period, the samples are then pulsed with radioactive ³H-thymidine and harvested. The uptake of thymidine is then measured using a β-spectrometer in order to determine whether cell-mediated immunity is present.

For purposes of the present invention, a four-fold increase in thymidine uptake for at least two of the samples above unstimulated cultures from the same PBMC or above antigen-stimulated cultures from the pre-immunization PBMC of the vaccinee (i.e., background levels) is considered to be an indication of the presence of cell-mediated protective immunity. The strength of such cell-mediated protective immunity would be proportional to the amount of such increase over background. For example, an eight-fold increase over background would be indicative of a stronger cell-mediated protective immunity than a four-fold increase.

The IL-2 assay for cell-mediated immunity also requires PBMC to be obtained from the vaccinee, preferably both before and after inoculation. Like the T-cell proliferation assay, the PBMCs are cultured in at least five different samples with media containing human plasma and separate HIV antigens for approximately seven days. Unlike the T-cell proliferation assay, though, anti-IL-2 receptor antibody is added to the cultures to block IL-2 consumption. At the end of the test period, the culture supernatants are harvested and assayed for IL-2 content using commercially available ELISA kits for the detection of IL-2. For purposes of the present invention, a four-fold increase in IL-2 production for at least two of the samples over unstimulated cultures from the same PBMC or above antigen-stimulated cultures from the pre-immunization PBMC of the vaccinee (i.e., background levels) will be considered to be an indication of the presence of cell-mediated protective immunity. As with the T-cell proliferation assay, a greater increase is indicative of a greater level of cell-mediated protective immunity.

A preferred manner of determining the presence or lack of an anti-HIV humoral immune response is with a conventional Enzyme Linked Immunosorbent Assay (ELISA). Suitable ELISA are commercially available from such companies as Organon-Technika, Ltd of Cambridge in the United Kingdom and Genetic Systems Corporation of Washington State. In both the Organon-Technika and Genetic Systems assays, whole HIV viruses are bound to a well of a microtiter plate. The assay is initiated by inserting blood serum from the vaccinee into the well. If anti-HIV antibodies are present in the serum, they will bind to the HIV present in the well. After these antibodies have had a chance to bind, the plate is washed. A labelled anti-antibody is subsequently added which will attach to any bound antibodies. After a second wash, the appropriate reagents for visualizing the label on the antibody are added. After an appropriate incubation period, the amount of antibodies present can be determined by measuring the optical density (“OD”) of the solution contained in each well.

In the Organon-Technika ELISA assay, an OD value of 0.20 or less is indicative of being seronegative for HIV antibodies and thus lacking a humoral response. By contrast, an OD value of 0.47 or greater in the organon-Technika ELISA assay or 2.4×background (i.e., 0.47 divided by 0.20) is indicative of being seropositive for HIV antibodies and thus having a humoral response. In the Genetic Systems ELISA assay, an OD value between 0.0 and 0.14 indicates the lack of a humoral response. By contrast, an OD value of 0.365 or greater in the Genetic Systems ELISA assay or 2.6×background (i.e., 0.365 divided by 0.14) indicates the presence of a humoral response.

Those skilled in the art recognize, of course, that there are other commercially available assays to determine either the presence of a cell-mediated response or the absence of a humoral response. Whatever assay one chooses, though, the vaccination objective of the present invention remains the same—to invoke a protective cell-mediated response without triggering an offsetting humoral response. To achieve this objective, a conservative low dose vaccination strategy should be adapted until sufficient feedback is obtained from follow-up assays to fine tune the vaccination dosages for subsequent inoculations. To the extent sufficient data has been collected from other vaccinees to fine tune the vaccination dosage in advance, a therapeutically effective dose of the vaccine can be provided to the vaccinee in the first instance, without the need for follow up assays.

Those of skill in the art will also recognize the applicability of the vaccination methods disclosed in this application to human lentiviruses generally [e.g., HIV-1, HIV-2, foamy virus (spumavirus)], simian lentiviruses (e.g., SIV) and other retroviruses (e.g., HTLV-I, HTLV-II).

To help illustrate the implementation of the present invention, the following examples are provided:

### EXAMPLE I

### Preparation of Attenuated HIV Immunogen With Nef Deletion

An attenuated HIV mutant is constructed by deleting part of the nef gene using standard deletion mutagenesis techniques. A 1.1 kb BamHI-SstI fragment from HIV cDNA clone BH10 [ATCC Accession No. 40125; see, Ratner et al., Nature 313: 277-284 (1985)] is isolated and subcloned into an M13mp18 expression vector. The vector is transformed into an E. coli host cell. Those transformants that contain the EcoRI fragment synthesizing a single-stranded DNA with the complementary nef coding sequence are identified. This single-stranded DNA is then isolated using standard techniques. See, e., Sambrook et al., Molecular Cloning: A Laboratory Manual, 2nd ed. Cold Spring Harbor Laboratory Press, 1989.

An oligonucleotide primer is synthesized that is complementary to the two regions flanking the portion of the nef gene that is to be deleted. For example, an oligonucleotide having the sequence 5′-ATA AGA CAG GGC TTG GAA AGG ATT TTG CTA TAA N CAT CGA GCT ACA AGG ACT TTC CGC TGG GGA ACT- 3′ is suitable for deleting the entire nef coding region, except for the portion of the nef gene that also codes for the 3′ end of the env gene. Nucleotides 8340-8994 [numbering system of Ratner et al., Nature 313: 277-284 (1985)] are deleted when this oligonucleotide is used for deletion mutagenesis.

oligonucleotide-directed mutagenesis is performed using the SCULPTOR™ Oligonucleotide-Directed in vitro Mutagenesis System, Version 2, from Amersham, Inc. (Arlington Heights, Ill.). The protocol is as described in the manufacturer's instructions, which are incorporated by reference. Briefly, the oligonucleotide primer is annealed to single-stranded DNA isolated from the E. coli transformants. The oligonucleotide primer is then extended using T7 DNA polymerase in the presence of dCTP's. The newly synthesize second strand is circularized using T4 DNA ligase.

The non-mutant strand, which does not contain the thiolated deoxynucleotide, is then removed. The double-stranded plasmid is digested with the restriction enzyme NciI, which does not cleave DNA strands that contain a thiolated nucleotide. The non-mutant strand is nicked by the enzyme. Exonuclease III is then utilized to extend the nick so that most of the non-mutant strand is degraded. Following this digestion, DNA polymerase I and T4 DNA ligase are used to synthesize a new strand, using the mutant strand as the template.

The resulting double-stranded clones are transformed into E, coli. Colonies that harbor the desired mutant fragment are identified and characterized by restriction mapping, DNA sequencing or other appropriate technique. The mutant fragment is then excised from the plasmid using BamHI and SstI endonucleases. The mutant fragment is used to replace the corresponding BamHI-SstI fragment in the wild-type HIV proviral clone. The newly constructed nef-deleted mutant is characterized to determine whether the correct deletion is present. After such characterization, the nef-deleted proviral clone is transformed into human T-cells for production of attenuated nef deleted HIV mutants in quantities needed for vaccines of the present invention.

### EXAMPLE II

### Preparation of gp160 Subunit HIV Immunogen

The gp160 subunit immunogen is prepared by heterologous expression in E. coli using standard recombinant techniques [Sambrook et al., Molecular Cloning: A Laboratory Manual, 2nd ed. Cold Spring Harbor Laboratory Press, 1989]. Briefly, the 2.7 kb KpnI fragment of HIV proviral clone BH10 (ATCC Accession No. 40125) is excised and purified. This fragment is cloned into a bacterial expression vector that contains a prokaryotic ribosome binding site (“RBS”) and an ATG initiation codon followed by a restriction site. For example, the pKK338-1 expression vector (Clontech Laboratories, Inc., Palo Alto, Calif.) is suitable. If necessary, an oligonucleotide linker is utilized to place the HIV KpnI fragment in the correct reading frame relative to the initiation codon of the expression vector. A linker can also be used to reconstruct the coding region for the 48-amino-terminal env amino acids that are not encoded by the KpnI fragment. The completed construct is checked by nucleotide sequencing, restriction analysis, or other appropriate technique. The vector is transformed into E. coli for expression.

Growth and expression of the E. coli transformants that contain the expression vector is carried out essentially as described in Sambrook et al., supra., Chapter 17, which is incorporated by reference. The recombinant gp160 protein is purified from the E. coli cells using standard purification techniques. See, e.g., Methods in Enzymology, Deutscher, M. P., ed., vol. 182, Academic Press, San Diego, 1990, which is incorporated by reference.

### EXAMPLE III

### Preparation of Immunogen Inactivated with Betapropriolactone

A 10 mg lot of gradient-purified HIV is diluted to 500 μg/ml (in 20 vials) and inactivated with 0.2% betapropiolactone (BPL) at 4° C. for 4 hours. Six μg from each treated vial is added to 1.5×10⁶ HUT-78 cells that had been pretreated for 1 hour with 2 μg/ml polybrene. After an overnight incubation (37° C., 5% CO₂), the cells are pelleted, washed three times, resuspended in fresh RPMI-1640 growth medium and distributed into culture flasks. Cultures are maintained with RPMI-1640 growth medium and kept at 37° C. and 5% CO₂ for 8 weeks. On days 14, 21, 28 and 56, clarified culture supernatants are assayed for p24 antigen concentration using a Coulter p24 antigen assay. If no activity is detected from this assay, the immunogen is suitable for use as a vaccine.

### EXAMPLE IV

### Preparation of Inactivated HIV Immunogen with Gag Deletion

A inactivated HIV mutant is constructed by deleting part of the gag gene that encodes the “zinc finger” region using standard deletion mutagenesis techniques. A 2.1 kb HindIII-KpnI fragment of HIV cDNA clone BH10 [ATCC Accession No. 40125; Ratner et al., Nature 313: 277-284 (1985)] is isolated and subcloned into an M13mp18 expression vector. The recombinant vector is transformed into E. coli and single-stranded DNA is isolated using standard techniques. See, e.g., Sambrook et al., Molecular Cloning: A Laboratory Manual, 2nd ed. Cold Spring Harbor Laboratory Press, 1989.

An oligonucleotide primer is synthesized that is complementary to the two regions that flank the portion of the zinc finger coding region that is to be deleted. For example, an oligonucleotide having the sequence 5′-AGA GGC AAT TTT AGG AAC CAA AGA AAG ATG GTT AAG GGC AAA GAA GGG CAC ACA GCC AGA AAT TGC AGG GCC-3′ is suitable for deleting 12 bp of the zinc finger coding region. Nucleotides 1507-1518 (numbering system of Ratner et al., supra.) are deleted when this oligonucleotide is used for deletion mutagenesis.

Importantly, the oligonucleotide maintains the correct reading frame for the remainder of the gag gene.

Oligonucleotide-directed mutagenesis is performed using the SCULPTOR™ Oligonucleotide-Directed in vitro Mutagenesis System, Version 2, from Amersham, Inc. (Arlington Heights, Ill.), as described in Example I.

The double-stranded clones that are obtained using the mutagenesis protocol are transformed into E. coli host cells. Colonies that harbor the desired mutant fragment are identified and characterized by DNA sequencing. The mutant fragment is then excised from the plasmid using KpnI and HindIII. This mutant fragment is used to replace the corresponding KpnI-HindIII fragment in the wild-type HIV proviral clone. The newly constructed zinc finger-deleted mutant is characterized to determine whether the correct deletion is present. After such characterization, the zinc finger-deleted proviral clone is transformed into human T-cells for production of sufficient inactivated HIV mutants for use as a vaccine.

### EXAMPLE V

### Preparation of Infectious HIV Immunogen

H9/HTLV-IIIB T-cell lines infected with specific HIV strains are obtained from the ATCC (ATCC Accession No. CRL 8543). The infected cells are thawed and cultured in RPMI media supplemented with 2.0 mM L-glutamine, 100 units/ml penicillin, 100 μg/ml streptomycin, 100 μg/ml gentamicin, 10% heat-inactivated fetal calf serum (Advanced Biotechnologies, Inc., Columbia, Md.) and 2 μg/ml polybrene (Sigma Chemical Co., St. Louis, Mo.) at 37° C. and 5% CO₂. Cells are counted every 3 to 4 days and adjusted to 5×10⁵ cells/ml. For the collection of the final viral stock, the cells are centrifuged at 250×g for 15 minutes, adjusted to 5×10⁵ cells/ml in the presence of fresh cell culture media and grown for an additional three days. Cells are in culture for a total of 14 days. The cells are then pelleted by centrifugation at 1000×g for 20 minutes, the supernatant is filtered through a 0.45 μm filter, adjusted to 20% fetal calf serum and frozen over liquid nitrogen in aliquots of ten-fold dilutions to form the final HIV virus stock. The pelleted cells are frozen for molecular characterization and as future reference samples.

### EXAMPLE VI

### Formulation of Vaccine with Infectious HIV Immunogen

Following the manufacturer's instructions, the concentration of HIV virus particles in the final HIV virus stock is determined using a Coulter HIV p24 Antigen Assay, whose instructions are incorporated by reference. Essentially, a control solution is first prepared by pipetting 200 μL normal human serum (“NHS”) or appropriate sample diluent into 5 microtiter wells coated with anti-p24-antibody. 50 μL of Antigen Reagent (Ag) are then added to 2 of the 5 control wells to provide 2 positive controls. 200 μL of each sample to be tested are pipetted into antibody-coated wells. 200 μL Lyse Buffer provided by Coulter are added to each well except the blank well. The plate is then securely sealed and incubated at 37° C. for 1 hour.

While incubation is occurring, a dilute 20×Wash Buffer is diluted 1× in distilled water and a CH-Biotin working solution is prepared. After incubation, the cover is removed from the plate and discarded. The solution is aspirated from each well and 300 μL of Wash Buffer provided by Coulter is added to each well. Aspiration and washing is then repeated five more times. After the final wash, the plate is inverted and tapped gently on a paper towel to remove any remaining liquid.

200 μL of CH-Biotin working solution is then added to each well, except the blank well. The plate is recovered and incubated for 1 more hour. During the incubation, an SA-HRPO working solution is prepared. After the second incubation, the aspiration and washing steps are repeated.

200 μL of diluted SA-HRPO working solution is then added to each well, except the blank well, and the plate is incubated again for 30 minutes at 37° C. During this incubation, a TMB substrate solution is prepared. The aspiration and washing steps are then repeated again and 200 μL of the TMB substrate solution are added to each well, except the blank well. After another incubation, 50 μL of CSR-1 solution is added to each well, including the blank well. The optical density (OD) of the plate is then read using a microtiter plate reader set at 450 nm.

By comparing the OD measurements against the standard p24 antigen concentration curve prepared as directed by the Coulter p24 assay instructions, the p24 gag antigen concentration of the final viral stock is determined. In order to obtain the desired concentration of 3 fg/ml, the final virus stock is diluted with physiologic saline. This diluted final virus stock is then ready for intramuscular inoculation in doses of 1 ml (i.e., 3 fg of p24 gag antigen per dose).

### EXAMPLE VII

### Formulation of Vaccine with Nef Deleted HIV Immunogen

Following the manufacturer's instruction as explained in the previous example, the Coulter HIV p24 Antigen Assay is used to determine the concentration of attenuated nef deleted virus in the final virus stock. In order to obtain the desired concentration of 100 fg/ml, the final virus stock is diluted with sterile 0.4% saline solution. This diluted final virus stock is then ready for intramuscular inoculation in doses of 1 ml (i.e., 100 fg of p24 gag antigen per dose).

### EXAMPLE VIII

### Formulation of Vaccine with gp160 Subunit Immunogen

The concentration of gp160 subunit immunogen from Example II is determined by methods known to one of skill in the art. See, e.g., Methods in Enzymology, vol. 182, supra, pp. 50-68 for several suitable methods. Immunological methods such as ELISA are also suitable, provided that a standard preparation of gp160 of known concentration is available to construct a standard concentration curve.

The concentration of gp160 in the preparation is adjusted to between 10 ng/ml and 10 μg/ml by diluting in sterile 0.4% saline solution. This diluted gp160 stock is then ready for intramuscular inoculation in doses of 1 ml (i.e., between 10 ng and 10 μg of gp160 per dose).

### EXAMPLE IX

### Administration of Vaccine Intramuscularly

The 1 ml gp160 subunit vaccine from Example VIII is administered intravenously to an adult weighing 150 lbs. using a syringe. Suitable sites for vaccination include the upper, outer quadrant of the gluteal area, the ventrogluteal area, the vastus lateralis of the thigh, or the deltoid muscle.

### EXAMPLE X

### Testing for Presence of Cell-mediated Immune Response

Testing for the presence of a sufficient cell-mediated immune response is done with an HIV antigen-stimulated T-Cell proliferation assay. Peripheral blood mononuclear cells (“PBMC”) are isolated from whole blood of the vaccinees immediately before the first immunization and approximately a month after the first immunization. Both PBMCs are cultured for a period of six days in five different samples at a concentration of 3×10⁵ cells per well in a volume of 0.2 ml RPMI 1640 culture media containing 5% human plasma and 2-5 μM of one selected HIV envelope antigen. The HIV envelope antigens selected for use in the five different samples are, respectively, gp160, T1, T2, P18-IIIB and P18-MN. Each culture is then pulsed with radioactive ³H-thymidine for 20 hours and harvested. The uptake of thymidine is measured using a β-spectrometer. If the uptake of thymidine from at least two of the PBMC samples taken approximately a month after the first immunization is found to be four or more times as great as the uptake of thymidine from the corresponding PBMC samples taken immediately before the first immunizations, it is concluded that the vaccination successfully induces a cell-mediated immune response.

### EXAMPLE XI

### Testing for Lack of Humoral Response

Testing for the lack of a humoral response is done with a whole virus ELISA kit obtained from Genetic Systems Corporation. Blood serum from the vaccinee is obtained immediately before the first immunization and three weeks after immunization. Each sample of blood serum is placed in a well in the Genetics Systems assay plate. After any antibodies from the blood serum have had an opportunity to bind to the whole HIV virus in the assay, the wells are washed. A labelled anti-antibody is added to each of the wells. After this labelled anti-antibody has had a sufficient opportunity to bind to any anti-HIV antibodies in the wells, the wells are washed again. The appropriate reagents for visualizing the label on the antibody are added. After an appropriate incubation period, the optical density of each sample is measured. If the measurements obtained for sample taken both before and after inoculation correspond to negative values for anti-HIV antibody, it is concluded that a humoral response has not been invoked from the vaccination.

### EXAMPLE XII

### Booster Inoculation to Elevate Level of Cell-mediated Response

After it is determined from a T-Cell proliferation assay that the first vaccination with nef deleted HIV vaccine of Example VII has created less than a four-fold increase of thymidine uptake over background, a booster inoculation program is appropriate. Accordingly, subsequent inoculations are made three times during weeks 0, 12 and 26 with the same nef deleted HIV vaccine of Example VII. Samples of PBMC are taken after each inoculation and tested in a T-Cell proliferation assay. If at the end of the 26th week, greater than a four-fold increase of thymidine over background is measured, further booster vaccinations are no longer necessary.

While the foregoing specification teaches the principles of the present invention, with examples provided for the purposes of illustration, it will be understood that the practice of the present invention encompasses all the usual variations, adaptations or modifications as come within the scope of the following claims and their equivalents.

What is claimed is:

1. A method for vaccinating a human against a human immunodeficiency virus comprising the steps of:
selecting an immunogen competent to induce a protective immune response in said human against said human immunodeficiency virus, and
administering to said human an effective amount of said immunogen sufficient to induce a sustained cell mediated immune response against said human immunodeficiency virus.

2. The method of claim 1 wherein said immunogen is an attenuated form of human immunodeficiency virus.

3. The method of claim 2 wherein said immunogen has been attenuated by removing all or part of the nef gene from the nucleic acid of said human immunodeficiency virus.

4. The method of claim 1 wherein said immunogen is a subunit of said human immunodeficiency virus.

5. The method of claim 4 wherein said immunogen is a gp120 subunit of said human immunodeficiency virus.

6. The method of claim 4 wherein said immunogen is a gp160 subunit of said human immunodeficiency virus.

7. The method of claim 1 wherein said immunogen is an inactivated human immunodeficiency virus.

8. The method of claim 7 wherein said immunogen has been inactivated by removing a sufficient portion of its genetic material so as to render it incapable of replicating.

9. The method of claim 8 wherein the genetic material removed from said human immunodeficiency virus is a portion of a gene coding for a gag nucleocapsid protein.

10. The method of claim 7 wherein said human immunodeficiency virus has been inactivated by exposure to a solution of betapropiolactone.

11. The method of claim 1 wherein said immunogen is an infectious form of human immunodeficiency virus administered in a subinfectious amount.

12. The method of claim 1 wherein the effective amount of immunogen administered contains between 100 attograms and 20 milligrams of p24 gag antigen.

13. The method of claim 2 wherein the effective amount of immunogen administered contains between 10 and 500 femtograms of p24 gag antigen.

14. The method of claim 11 wherein the effective amount of immunogen administered contains between 100 attograms and 500 femtograms of p24 gaq antigen.

15. The method of claim 1 wherein a cell mediated response is determined to be present using a T-Cell proliferation assay if the uptake of thymidine by antigen-stimulated cells is at least four-fold above background.

16. The method of claim 1 wherein a cell mediated response is determined to be present using an IL-2 assay if the production of IL-2 by antigen-stimulated cells is at least four-fold above background.

17. A method for vaccinating a human against a human immunodeficiency virus comprising the steps of:
selecting an immunogen competent to induce a protective immune response in said human against said human immunodeficiency virus, and
administering an effective amount of said immunogen to said human sufficient to induce a cell mediated response against said human immunodeficiency virus but below the amount necessary to induce a humoral response.

18. A method for vaccinating a human against a mammalian retrovirus comprising the steps of:
selecting an immunogen competent to induce a protective immune response in said mammal against said retrovirus, and
administering an effective amount of said immunogen to said mammal sufficient to induce a cell mediated immune response against said retrovirus but below the level necessary to induce a humoral response.

19. The method of claim 18 wherein said retrovirus is a simian immunodeficiency virus.

20. The method of claim 18 wherein said mammal is a human.

21. The method of claim 20 wherein said retrovirus is HTLV-I.

22. The method of claim 20 wherein said retrovirus is HTLV-II.

23. The method of claim 20 wherein said retrovirus is foamy virus.

24. A vaccine comprising a therapeutically effective dose of an immunogen capable of eliciting a cell-mediated immune response in a human protective against infection by a human immunodeficiency virus.

25. A vaccine comprising a dose of immunogen capable of eliciting a cell-mediated response in a human as measured by a T-cell proliferation assay.
